(19) United States
(12) Patent Application Publication
(10) Pub. No.: US 2024/0074899 A1
(43) Pub. Date: Mar. 7, 2024

(54) Silicone Oil-Induced Glaucomatous Neurodegeneration in Non-human Primate

(71) Applicant: The Board of Trustees of the Leland Stanford Junior University, Stanford, CA (US)

(72) Inventor: Yang Hu, Palo Alto, CA (US)

(21) Appl. No.: 18/376,927

(22) Filed: Oct. 5, 2023

Related U.S. Application Data
(63) Continuation-in-part of application No. 17/420,887, filed on Jul. 6, 2021.
(60) Provisional application No. 62/795,234, filed on Jan. 22, 2019.
(60) Provisional application No. 63/415,169, filed on Oct. 11, 2022.

(51) Int. Cl. A61F 9/007; G09B 23/30

(52) U.S. Cl. CPC A61F 9/00781; G09B 23/306

(57) ABSTRACT

Injection of silicon oil (SO) to the anterior chamber of an eye efficiently induces intraocular pressure (TOP) elevation. This effect occurs without causing overt ocular structural damage or inflammatory responses while simulating acute glaucomatous changes that human patients develop over years by inducing progressive RGC and ON degeneration and visual functional deficits within weeks. The anterior segments of the experimental eyes are not substantially affected, leaving clear ocular elements that allow easy and reliable assessment of in vivo visual function and morphology. More importantly, this is the only reversible ocular hypertension model by removing SO from the anterior chamber and particularly useful for testing neuroprotection treatment together with lowering TOP treatment. In summary, the acute ocular hypertension glaucoma model replicates secondary post-operative glaucoma. It is straightforward and reversible, does not require special equipment or repeat injections, and may be applicable to a range of animal species with only minor modifications.

### CROSS-REFERENCE TO RELATED APPLICATIONS

This application claims priority from U.S. Provisional Patent Application 63/415,169 filed Oct. 11, 2022, which is incorporated herein by reference. This application is a continuation-in-part of U.S. patent application Ser. No. 17/420,887 filed Jul. 6, 2021, which is incorporated herein by reference. U.S. patent application Ser. No. 17/420,887 is a 371 of PCT application PCT/US2020/013958 filed Jan. 16, 2020. PCT application PCT/US2020/013958 claims the benefit of U.S. Provisional application 62/795,234 filed Jan. 22, 2019.

### STATEMENT OF GOVERNMENT SPONSORED SUPPORT

This invention was made with Government support under contract EY031063 awarded by the National Institutes of Health. The Government has certain rights in the invention.

### FIELD OF THE INVENTION

This invention relates to methods, devices and systems for the treatment of glaucoma.

### BACKGROUND OF THE INVENTION

Glaucoma is the most common cause of irreversible blindness and will affect more than 100 million individuals between 40 and 80 years of age by 2040. Annual direct medical costs to treat this disease in 2 million patients in the United States totaled $2.9 billion. Glaucoma is a neurodegenerative disease characterized by injury to the axons of retinal ganglion cells (RGCs) followed by progressive degeneration of RGC somata and axons within the retina and Wallerian degeneration of the myelinated axons in the optic nerve (ON). The level of intraocular pressure (IOP) is the most common risk factor. Current clinical therapies target reduction of TOP to retard glaucomatous neurodegeneration, but neuroprotectants are critically needed to prevent degeneration of RGCs and ON.

Similar to other chronic neurodegenerative diseases, the search for neuroprotectants to treat glaucoma continues. To longitudinally assess the molecular mechanisms of glaucomatous degeneration and the efficacy of neuroprotectants, a reliable, reproducible, and inducible experimental ocular hypertension/glaucoma model is essential.

Rodents serve as a mammalian experimental species of choice for modeling human diseases and large-scale genetic manipulations. Various rodent ocular hypertension models have been developed including spontaneous mutant or transgenic mice and rats and mice with inducible blockage of aqueous humor outflow from the trabecular meshwork (TM). While genetic mouse models are valuable to understand the roles of a specific gene in TOP elevation and/or glaucomatous neurodegeneration, the pathologic effects may take months to years to manifest. Inducible ocular hypertension that develops more quickly and is more severe term would be preferable for experimental manipulation and general mechanism studies, especially for neuroprotectant screening. Injection of hypertonic saline and laser photocoagulation of the episcleral veins and TM are commonly used in rats and larger animals. Although similar techniques also produce ocular hypertension in mice, they are technically challenging, and irreversible ocular tissue damage and intraocular inflammation complicate their interpretation. Intracameral injection of microbeads to occlude aqueous humor circulation through TM produces excellent TOP elevation and glaucomatous neurodegeneration. However, retaining microbeads at the angle of the anterior chamber and controlling the degree of aqueous outflow blockade are difficult. Furthermore, its lengthy duration (6-12 weeks after microbeads injection) causes death of only less than 30% of RGC, leaving a narrow window for preclinical testing of neuroprotective therapies. It is therefore critically important to develop an effective ocular hypertension model that closely resembles human glaucoma, and that can be readily adapted among different species with minimal confounding factors.

Secondary glaucoma with acutely elevated TOP occurs as a post-operative complication following the intravitreal use of silicone oil (SO) in human vitreoretinal surgery. SO is used as a tamponade in retinal detachment repair because of its buoyancy and high surface tension. However, SO is lighter than the aqueous and vitreous fluids and an excess can physically occlude the pupil, which prevents aqueous flow into the anterior chamber. This obstruction increases aqueous pressure in the posterior chamber and displace the iris anteriorly, which causes angle-closure, blockage of aqueous outflow through TM, and a further increase in TOP. Prophylactic peripheral iridotomy that maintains the circulation between anterior and posterior chambers normally prevents this type of secondary glaucoma.

### SUMMARY OF THE INVENTION

A reliable glaucoma model that closely mimics the disease in humans is a prerequisite for studies of pathogenetic mechanisms and for selecting efficient neuroprotective treatments for clinical use. In the present invention, such a highly effective and reproducible model and method was developed. Injection of SO to the mouse anterior chamber efficiently induces a series of reactions, including pupillary block, blockage of the aqueous humor outflow from anterior chamber, accumulation of aqueous humor in the posterior chamber, closure of the anterior chamber angle, and TOP elevation. These reactions occur without causing overt ocular structural damage or inflammatory responses while simulating acute glaucomatous changes that human patients develop over years by inducing progressive RGC and ON degeneration and visual functional deficits within weeks.

SO injection is limited to one eye (experimental eye) in each mouse, with the other eye (contralateral eye) receiving an equivalent volume of normal saline. This serves as a convenient internal control for the surgical procedure and for studies of RGC morphology and function. It is reasonable to conclude that TOP is elevated in the SOHU eyes because of impeded inflow and accumulation of aqueous humor in the posterior segment of the eye, rather than by an aspect of the surgical procedure, such as the cornea wound or inflammation, which was rare.

Because of the unique feature of pupillary block associated with SOHU, the TOP is elevated in the posterior part of the eye, but not in the anterior chamber. The inventors postulated that, after the pupil is sealed by SO, the large mouse lens, together with the iris and ciliary body, forms a rigid barrier that essentially disconnects the anterior and posterior chambers and thus shields the anterior chamber from the high pressure in the posterior chamber. This pathogenesis gives the model two advantageous characteristics: 1) The anterior segments of the experimental eyes are not substantially affected, leaving clear ocular elements that allow easy and reliable assessment of in vivo visual function and morphology; 2) The high TOP of the posterior chamber causes pronounced glaucomatous neurodegeneration within 5-8 weeks, which facilitates testing neuroprotectants by allowing any benefit to be detected in a short period of experimental time.

Understanding the molecular mechanism of glaucoma and development of neuroprotectants are significantly hindered by the lack of a reliable animal model that accurately recapitulates human glaucoma. In this invention, we developed a model for the secondary glaucoma that is often observed in humans after silicone oil (SO) blocks the pupil or migrates into the anterior chamber following vitreoretinal surgery. We observed similar intraocular pressure (TOP) elevation after intracameral injection into mouse eyes of SO, and removing the SO allows the TOP level to quickly return to normal. This inducible and reversible model showed dynamic changes of visual function that correlate with progressive RGC loss and axon degeneration. We also used a single AAV vector for the first time to co-express miRNA-based shRNA and a neuroprotective transgene and further to validated this model as an effective in vivo means to test neuroprotective therapies by targeting neuronal endoplasmic reticulum stress.

Embodiments of this invention and model can be adapted to other experimental animal species to produce stable, robust TOP elevation and significant neurodegeneration. The model produces standardized ocular hypertension-induced pathology and supports studies of pathogenetic mechanisms and of selection of neuroprotectants for glaucoma.

The invention is embodiment as a model or device as a silicone oil-induced ocular hypertension glaucoma model distinguishing an experimental eye with an anterior chamber having in the anterior chamber a silicone oil droplet larger than 1.5 mm in diameter. The silicone oil droplet is equivalent to about 1-2 microliters. The model could be enhanced by a contralateral eye with an anterior chamber having in the anterior chamber a volume of saline which is used as a control eye relative to the experimental eye. The volume of saline is equivalent to about 1-2 microliters.

The invention is embodiment as a method of modeling intraocular hypertension distinguishing the steps of injecting into an anterior chamber of an experimental eye a silicone oil to form a droplet of at least 1.5 mm in diameter inside the anterior chamber. The injected silicone oil is equivalent to about 1-2 microliters. The method could further distinguish injecting into an anterior chamber of a contralateral eye a volume of saline which is used as a control eye relative to the experimental eye. The volume of saline is equivalent to about 1-2 microliters.

In one embodiment, the model is based on an animal, and it this teaching, specifically, a mouse model was used, however, the particular animal model is not limited to mice as it could also be a primate model or any other animal model that closely mimics the human eye anatomy and physiology.

Understanding the molecular mechanism of glaucoma-induced neurodegeneration and development of neuroprotectants are significantly hindered by the lack of a reliable large animal model that accurately recapitulates human glaucoma.

Because the anatomy of the non-human primate (NHP) visual system closely resembles that of humans, it is the most likely to predict human responses to diseases and therapies. Here the inventor replicated its original mouse SOHU glaucoma model in NHP, rhesus macaque monkeys.

In one embodiment, a disposable 15-degree blade was used to make a side-port incision at the corneal limbus to enter the anterior chamber inferiorly near the 6 o'clock position to minimize the likelihood of oil leaking out of the eye. Silicone Oil (SO, 1,000 mPa·s, Silikon, Alcon Laboratories, Fort Worth, Texas) in a 3 cc syringe on a bent 25 gauge cannula was introduced into the anterior chamber. SO was injected little by little, stopping intermittently with gentle pressure applied to the posterior aspect of the limbal incision to allow for aqueous humor to exit the eye. Oil was injected to fill the anterior chamber to a physiologic depth with roughly ˜70-80% silicone oil and to cover the entire pupil with ˜100 μl volume. This acute NHP glaucoma model closely recapitulates the major features of glaucomatous neurodegeneration in humans, all the tested animals showed significant retinal ganglion cell (RGC) death, optic nerve degeneration, and visual functional deficits at both 3 and 6 months. It is therefore suitable for studying the pathology of primate RGC/ON, assessing experimental therapies for neuroprotection and regeneration, and therefore for translating relevant findings into novel and effective treatments for patients with glaucoma and other neurodegenerations.

This model can be used and adapted by pharmaceutical and biotech companies for the selection of neuroprotectants for glaucoma.

The current NHP glaucoma model is using laser to damage anterior segment of the eye, which is difficult to perform and causes irreversible damage but variable neurodegeneration phenotype. The SO glaucoma model is easier to perform with stable and significant neurodegeneration phenotype.

The volume of the droplet ranges from 75-150 μl, which results in a size of the droplet to cover 70-80% of the anterior chamber. These ranges are larger than the ones used for the mouse model simply due to the fact that monkeys are larger animals than mice. In any case, the droplet size is such that the droplet/bulb covers the pupil.

The description herein teaches what SO is doing to cause TOP elevation. It was demonstrated that SO blocks the pupil to prevent aqueous humor migration from the back of the eye into anterior chamber to increase IOP. 2) Embodiments of this invention do not require a combination of an SO injection with electrocoagulation of limbal vessels. The approach presented herein does not require the irreversible damage of limbal vessels.

In another embodiment, the invention can be described as a silicone oil-induced ocular hypertension glaucoma model in a living non-human primate. The model has an experimental eye with an iris, a pupil and an anterior chamber having in the anterior chamber a silicone oil droplet covers 70-80% iris surface, where the silicone oil droplet is equivalent to about 75-150 microliters and closes the anterior chamber angle, and where the silicone oil contacts the surface of the iris such that pupillary block occurs.

The silicone oil-induced ocular hypertension glaucoma model further has a contralateral eye with an anterior chamber having in the anterior chamber a volume of saline which is used as a control eye relative to the experimental eye. The volume of saline is equivalent to about 75-150 microliters.

### BRIEF DESCRIPTION OF THE DRAWINGS

Some of the drawings had to be converted from color images to grey scale images with the result that some of the details might have gotten lost. The reader is referred to U.S. Provisional Patent Application 63/415,169 filed Oct. 11, 2022 for review the original drawings if desired.

FIG. 1 shows according to an exemplary embodiment of the invention intracameral SO injection pupil blocking. The equipment used for in vivo assays, including SLO/OCT, ERG, fundus imaging, and TonoVet for TOP measurement.

FIGS. 2A-C shows according to an exemplary embodiment of the invention visual function and morphological deficits of SOHU monkey eyes. (FIG. 2A) Longitudinal SD-OCT imaging of SOHU retinas at inferior (I), superior (S), nasal (N), and temple (T) quadrants; and the H&E staining of retina sections. (FIG. 2B) Measurements of RNFL thickness at different time points, represented as percentage of SOHU eyes compared to CL eyes. Data are presented as means±s.e.m, n=6 for 3 mpi and n=4 for 6 mpi, *: P<0.05, **: P<0.01, ***: P<0.001, Student's t test. (FIG. 2C) Longitudinal ERG recording of macaque eyes at different time points after SO injection and the measurements of the amplitudes of a wave, b wave and PhNR, represented as percentage of the amplitudes in the SOHU eyes, compared to the CL eyes. Data are presented as means±s.e.m, n=6 for 1-3 mpi and n=4 for 4-6 mpi, *: P<0.05, **: P<0.01, One-way ANOVA with Tukey's multiple comparison test.

FIGS. 3A-D show according to an exemplary embodiment of the invention severe RGC and ON degeneration in SOHU eyes at 3 mpi and 6 mpi. (FIG. 3A) Confocal images of wholemount retinas showing surviving RBPMS-positive (red) RGCs in the peripheral, mid-peripheral, and central retina at 3 and 6 mpi. Scale bar, 20 μm. (FIG. 3B) Quantification of surviving RGCs in the peripheral, mid-peripheral, and central retina. CL: contralateral control eyes. Data are presented as means±s.e.m, n=2 for 3 mpi and n=4 for 6 mpi, ***: P<0.001, ****: P<0.0001, one-way ANOVA with Tukey's multiple comparison test. (FIG. 3C) Light microscope images of semi-thin transverse sections of ON stained with PPD in the corresponding groups. Upper panel: 100×, Scale bar, 20 μm; lower panel: 60×, Scale bar, 500 μm. (FIG. 3D) Quantification of surviving RGC axons in ON. Data are presented as means±s.e.m, n=2 for 3 mpi and n=4 for 6 mpi, **: P<0.01, ***: P<0.001, One-way ANOVA with Tukey's multiple comparison test.

FIGS. 4A-B show according to an exemplary embodiment of the invention dynamic TOP changes of SOHU eyes. Presentations of longitudinal IOP measurements of experimental (SOHU) eyes and contralateral control (CL) eyes at different time points after SO injection. mpi: month post injection. Data are presented as means±s.e.m, n=6 (1-3 mpi) and n=4 (4-6 mpi) of each group; *: p<0.05, ***: p<0.001, Student's t test.

FIG. 5 shows according to an exemplary embodiment of the invention ciliary body atrophy in SOHU eyes at 6 mpi. Anterior chamber sections stained with H&E and imaged with 2× lens; and enlarged images of ciliary body, showing loose arrangement, larger interfibrous areas, and increased cellular invasion in muscle fibers.

FIGS. 6A-C show according to an exemplary embodiment of the invention ONH “cupping” in animal #38361 associated with TOP elevation. (FIG. 6A) The retinal fundus images of the SOHU eye before and after SO injection. ‘Yellow’ dotted line outlines the optic disc; ‘blue’ dotted line outlines optic cup. (FIGS. 6B-C) Longitudinal SD-OCT imaging of macaque ON head with 48 radial B-scans acquired over a 30° area at 768 A-scans per B-scan, ART=16 repetitions.

### DETAILED DESCRIPTION

The present invention is a method and model developed as a procedure for intracameral injection of silicone oil (SO) to block the pupil, which causes acute ocular hypertension and significant retinal ganglion cell (RGC) and optical nerve (ON) degeneration. The present invention demonstrates that embodiments of this invention, which may be adaptable to different species, induces stable intraocular pressure (TOP) elevation and profound neuronal response to ocular hypertension in the retina that will expedite selection of neuroprotectants and establishing the pathogenesis of acute ocular hypertension-induced glaucoma. First, the materials and methods will be discussed, after which results of using the method and model will be presented. For the purpose of model development and testing of the methodology a non-human primate model was used.

Intracameral Injection of SO in Rhesus Macaque Monkey Causes RNFL Thinning and Decreases PhNR

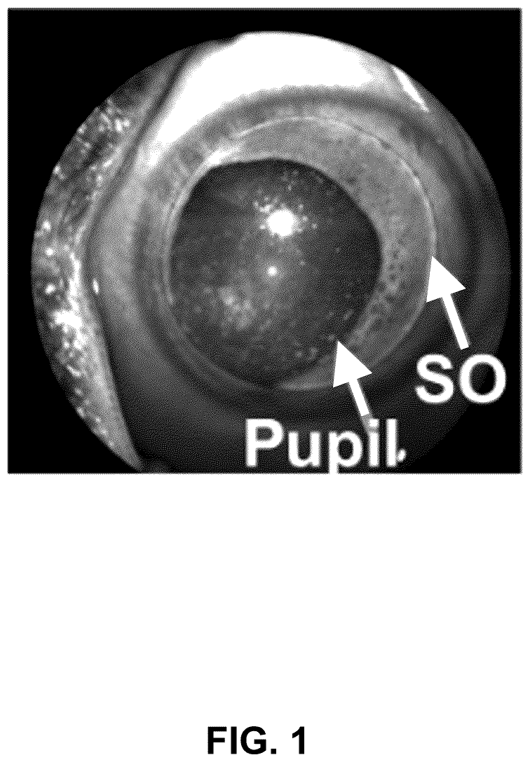

We injected roughly 100 μl SO into the anterior chamber of the right eyes of 6 macaque monkeys (reference is made to Table 1 in U.S. Provisional Patent Application 63/415,169 filed Oct. 11, 2022, which is incorporated herein by reference), filling 80% SO of the anterior chamber with complete covering of the pupil (FIG. 1).

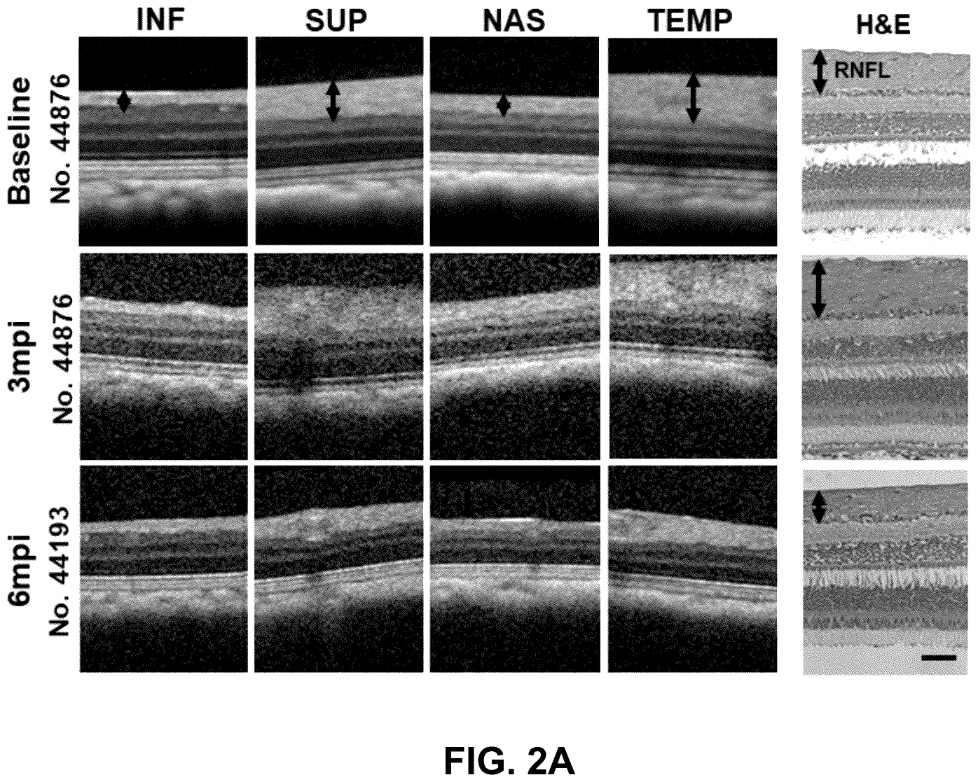

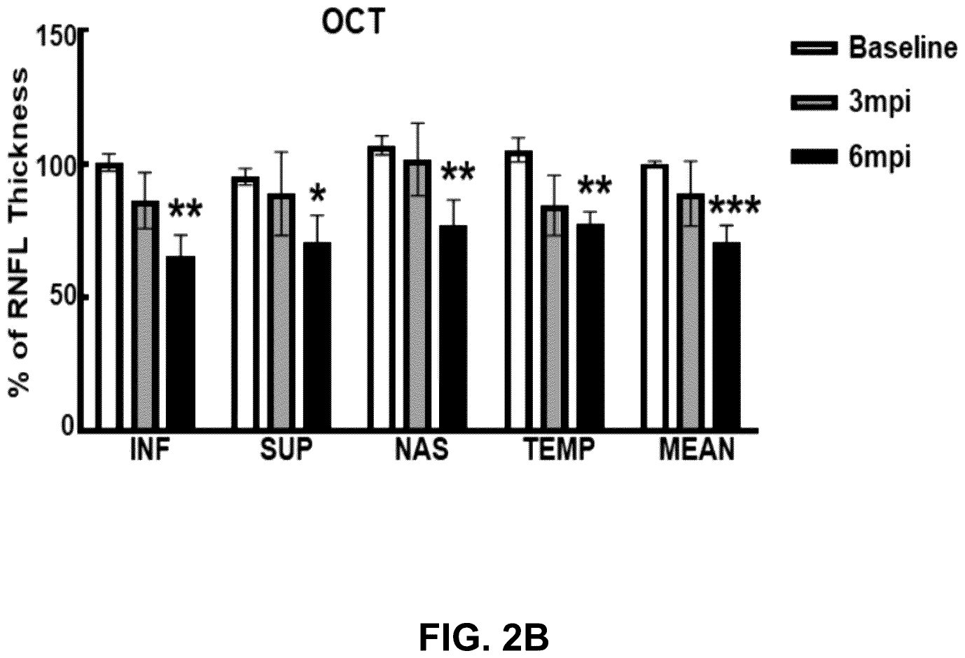

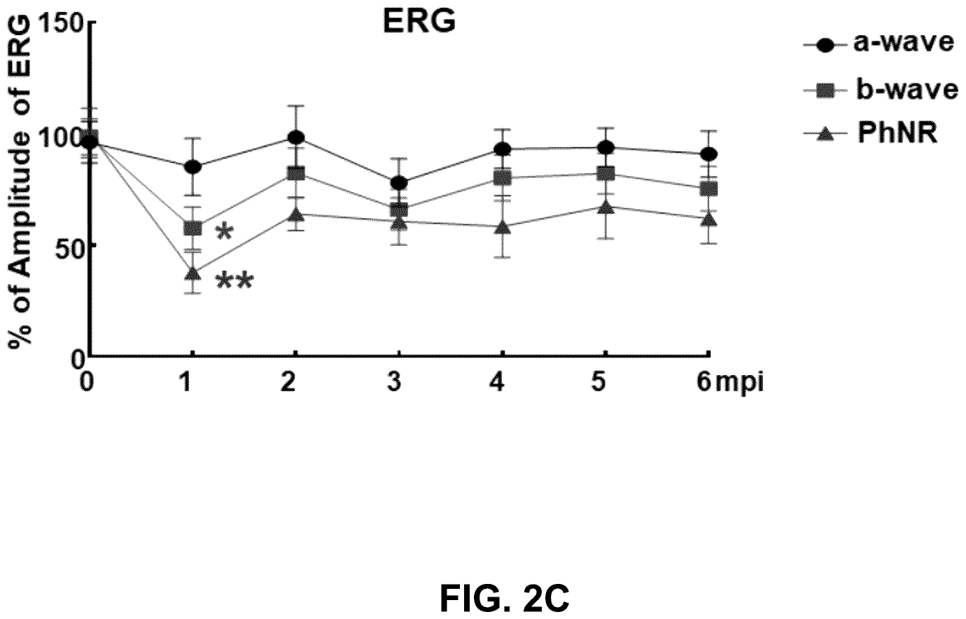

Retinal morphology and function were assayed before SO injection and at different time points after. These assays included fundus imaging, spectral-domain optical coherence tomography (SD-OCT), and electroretinography (ERG). Thinning of the retina nerve fiber layer (RNFL) measured by OCT is used clinically as a biomarker for RGC/ON degeneration. We measured the RNFL thickness of the animals and detected edema (thickening) of RNFL in the SOHU eyes at 3-month post injection (3 mpi) and significant thinning at 6 mpi (FIGS. 2A-B), indicating inner retina neurodegeneration. We also examined the visual function of these macaques. The photopic negative response (PhNR) of the photopic full-field ERG is a negative-going wave that occurs after the b-wave in response to a brief flash and reflects the function of RGCs and their axons in general. Its amplitude is reduced early in human glaucoma, which also correlates well with structural loss in NHP glaucoma. Both b-wave and PhNR's amplitudes decreased in the SOHU eyes at all time points after SO injection, but only reached statistical significance at 1 mpi (FIG. 2C), suggesting functional deficits of the inner retina.

Significant RGC and ON Degeneration of the SOHU Eyes at 3 Mpi and 6 Mpi in all Tested Animals

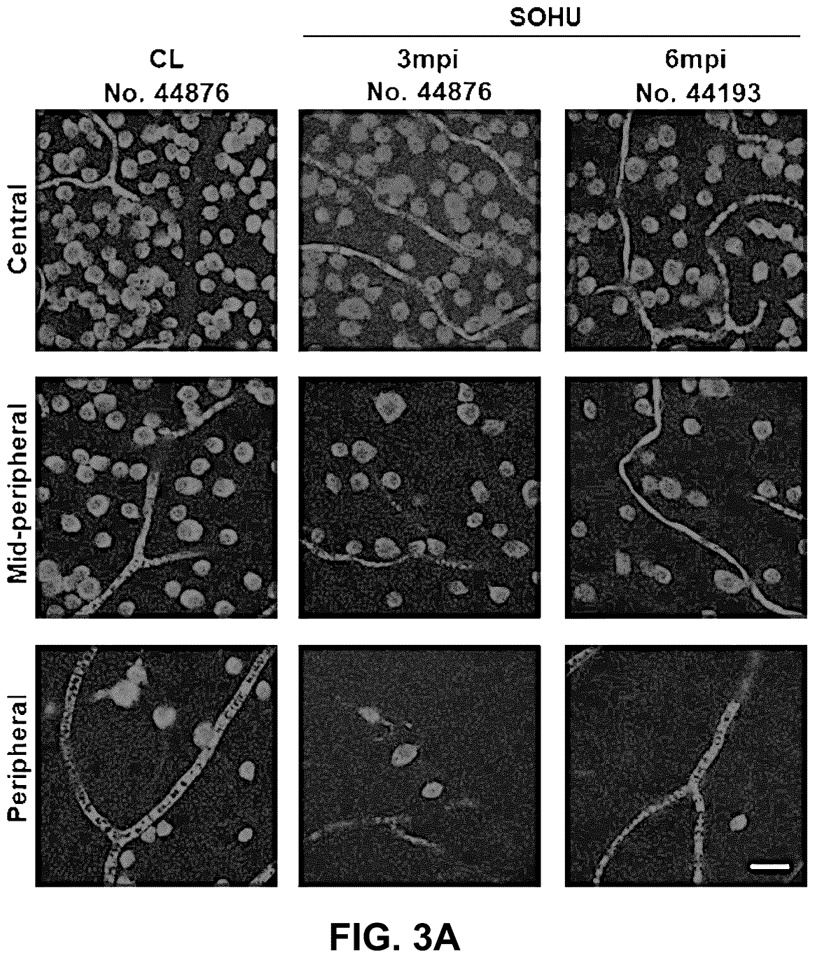

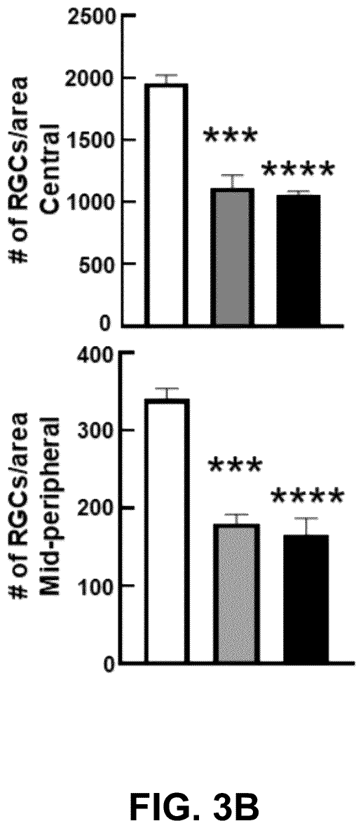

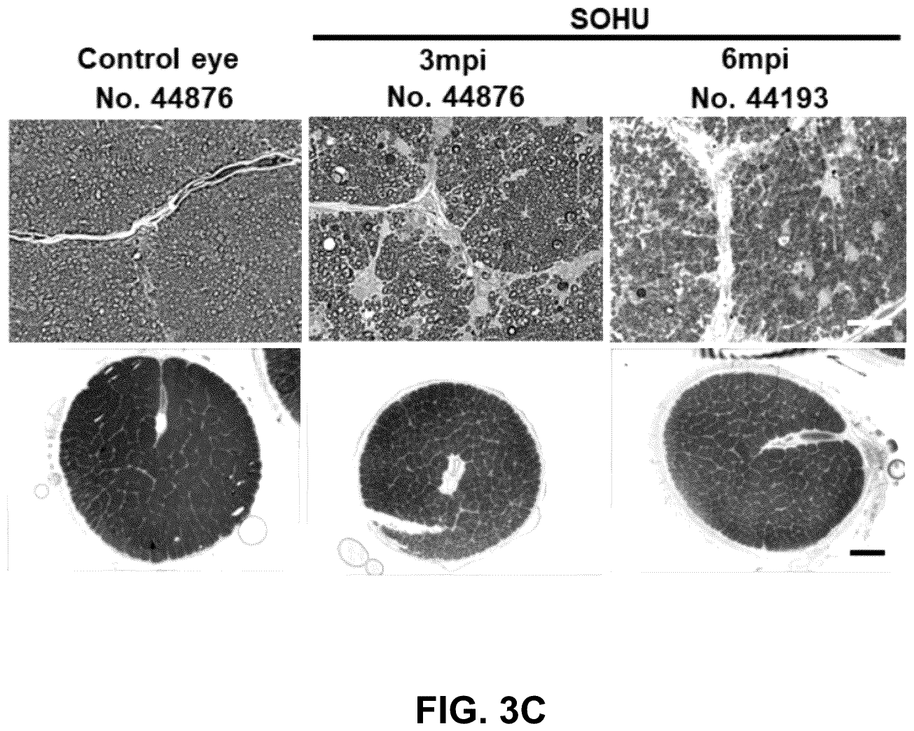

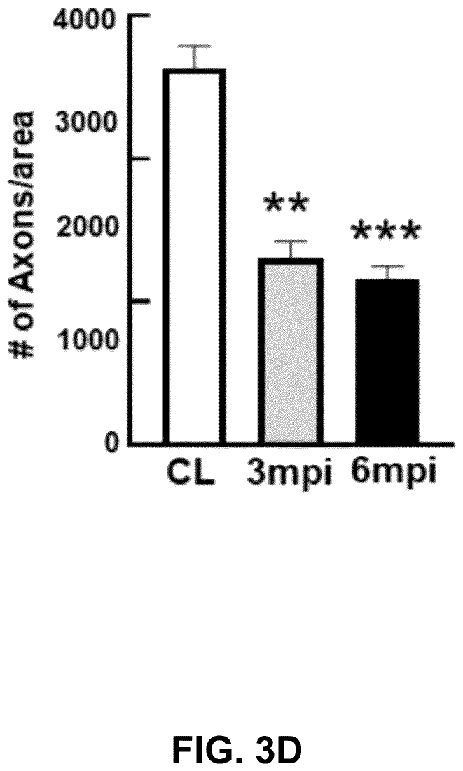

To confirm the glaucomatous neurodegeneration, we euthanized two animals at 3 mpi and four animals at 6 mpi for histological analysis of post-mortem retina and ON. Consistent with the in vivo structural and functional deficits detected in the living animal, retinal wholemounts revealed significant RGC somata loss in the SOHU eye throughout the peripheral to the central retinas at both 3 mpi and 6 mpi (FIGS. 3A-B); and semithin cross-sections showed significant RGC axon degeneration in ON at both 3 mpi and 6 mpi (FIGS. 3C-D), indicating significant glaucomatous neurodegeneration of the SOHU eyes.

Dynamic IOP Changes in the SOHU Macaque Eyes Associated with Ciliary Body Atrophy

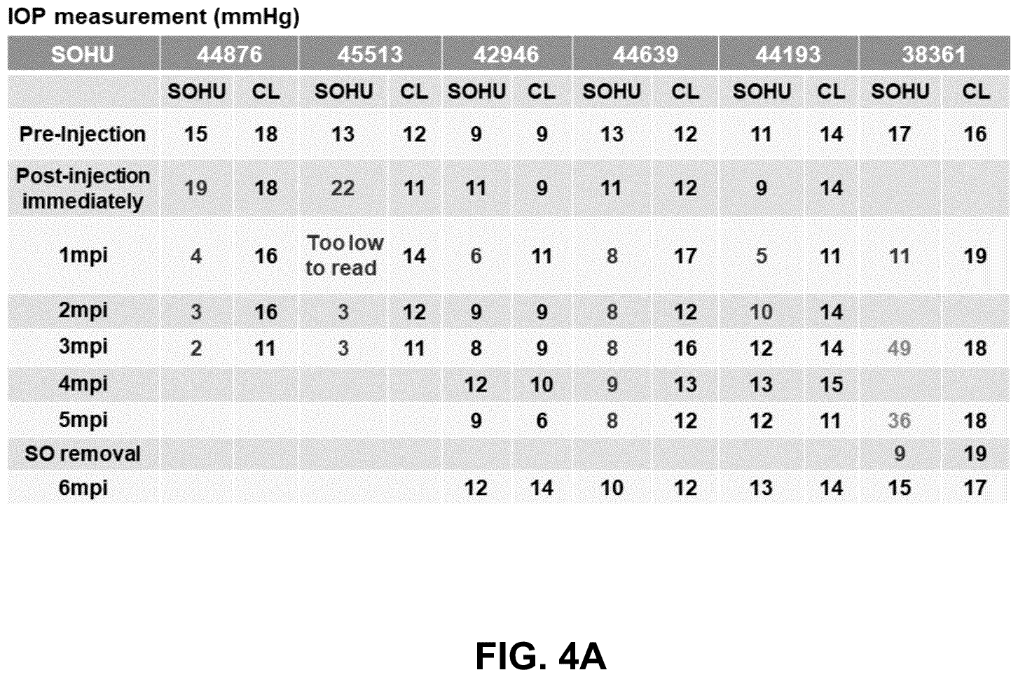

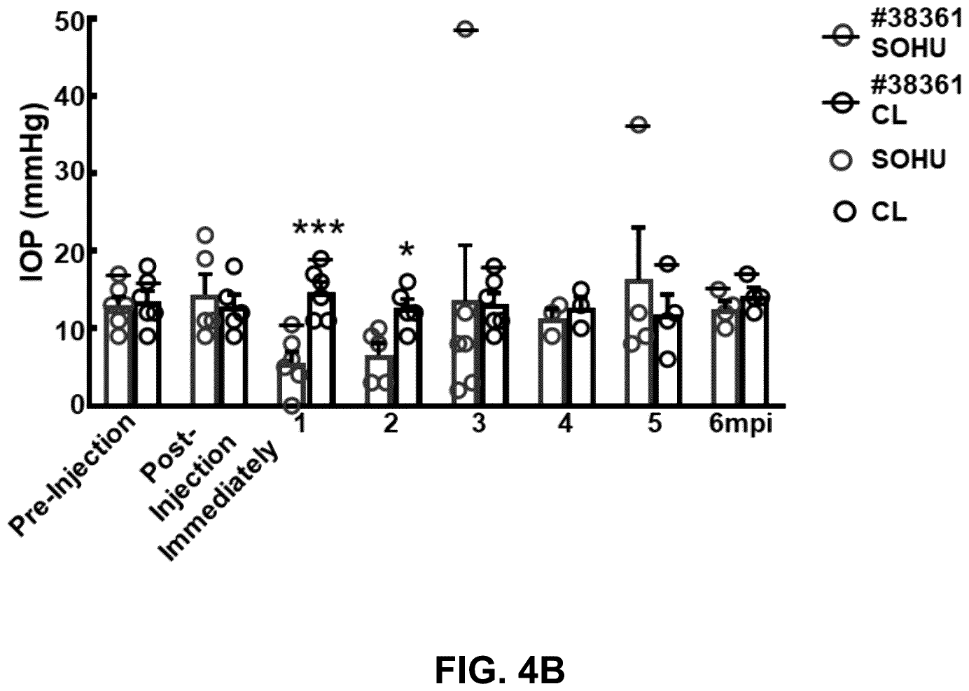

Surprisingly, these macaques showed different IOP dynamics after SO injection. In two animals (#44876 and #45513), IOP was elevated immediately after SO injection (15 to 19 mmHg and 13 to 22 mmHg, FIG. 4A). Because restrictions of the Primate Center then precluded measuring the IOPs before 1 mpi or more frequently than once a month thereafter, we could not measure the IOP earlier or more often. Therefore, we do not know for the duration of the transient IOP elevation after SO injection. However, all six animals showed substantial ocular hypotension at 1 mpi and 2 mpi: IOPs of the SOHU eyes were much lower than their baselines or their contralateral control eyes (FIGS. 4A-B). The ocular hypotension lasted from 1 mpi to 3 mpi in two animals (#44876 and #45513) and from 1 mpi to 5 mpi in one animal (#44639); IOP returned progressively to normal between 2-6 mpi in three animals (#42946, #44639, and #44193) that we maintained for 6 months.

In one animal (#38361) IOP was much higher than normal from 3-5 mpi, at first fell significantly when SO was removed from the eye at 5 mpi, then returned to normal one month later. The sequence of changes in this animal indicated that the SO-induced pupillary blocking was the cause of TOP elevation, and that simply removing the SO reversed the pupillary blocking and ocular hypertension.

Because we missed the measurement at the 2 mpi time point for this animal (#38361), we assume that the TOP of the SOHU eye recovered from ocular hypotension and became elevated between 1 mpi and 3 mpi.

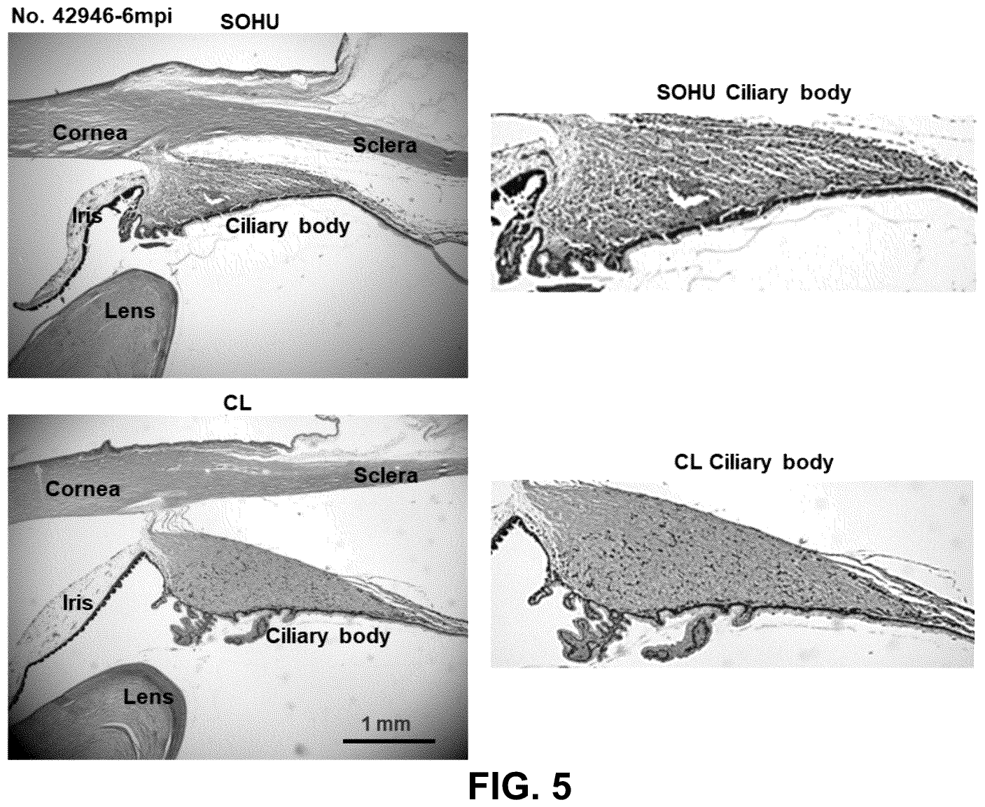

We suspect that the pupillary blockade caused a substantial elevation of the TOP acutely, which led to ciliary body “shutdown”, as in some human patients. The subsequent lasting ocular hypotony then happened due to ceased aqueous production from ciliary body. Indeed, the ciliary body was severely atrophied in the SOHU eyes of all animals, revealed by H&E staining of the anterior segments of the eyes (FIG. 5). There was no inflammation or obvious deformation of cornea, sclera, iris, or lens, although the pupils of the SOHU eyes were fixed in the mid-dilated state, suggesting a transient high TOP elevation, which may result in ischemic iris sphincter muscle and consequently limitation in constriction, as in patients with acute angle closure glaucoma.

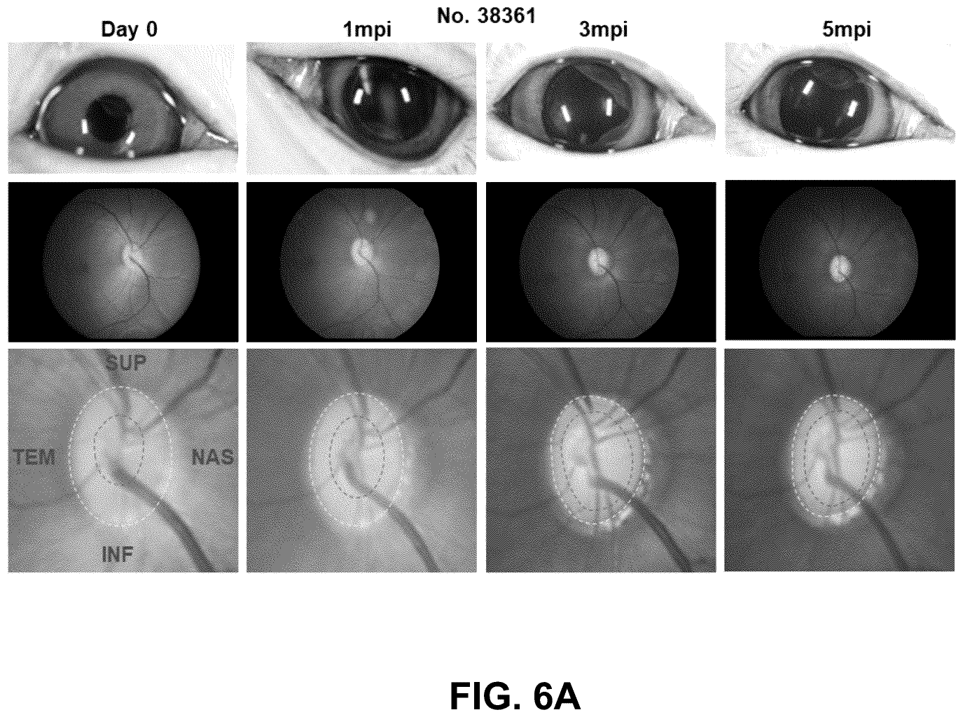

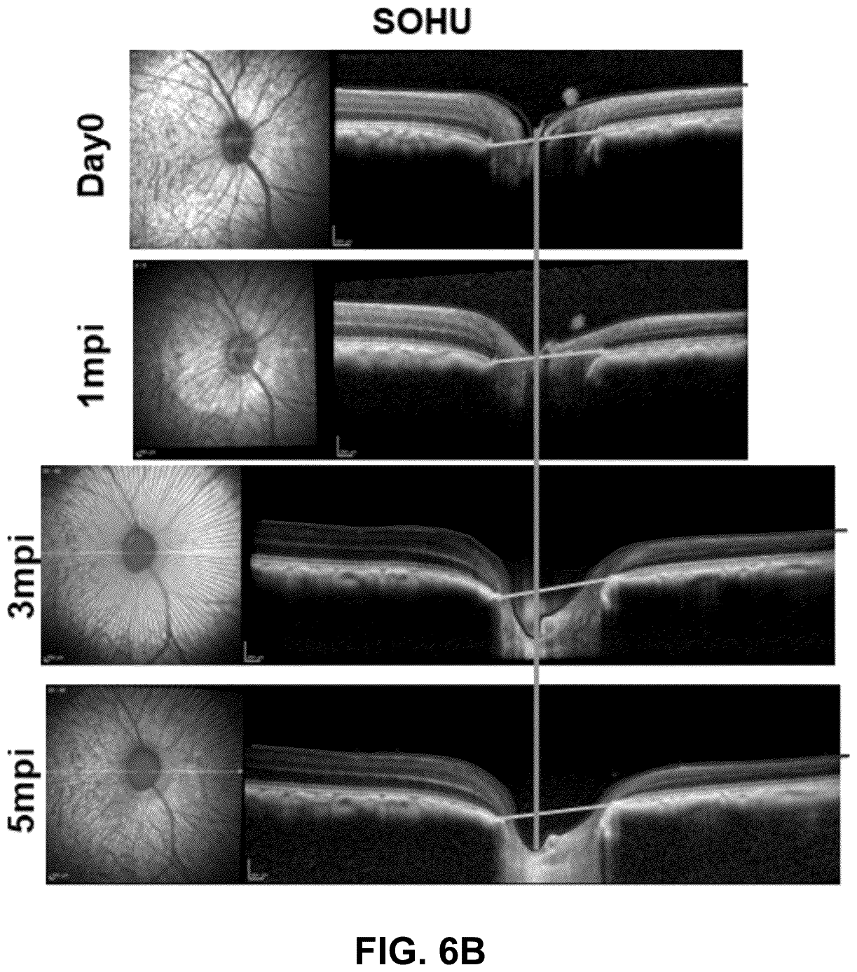

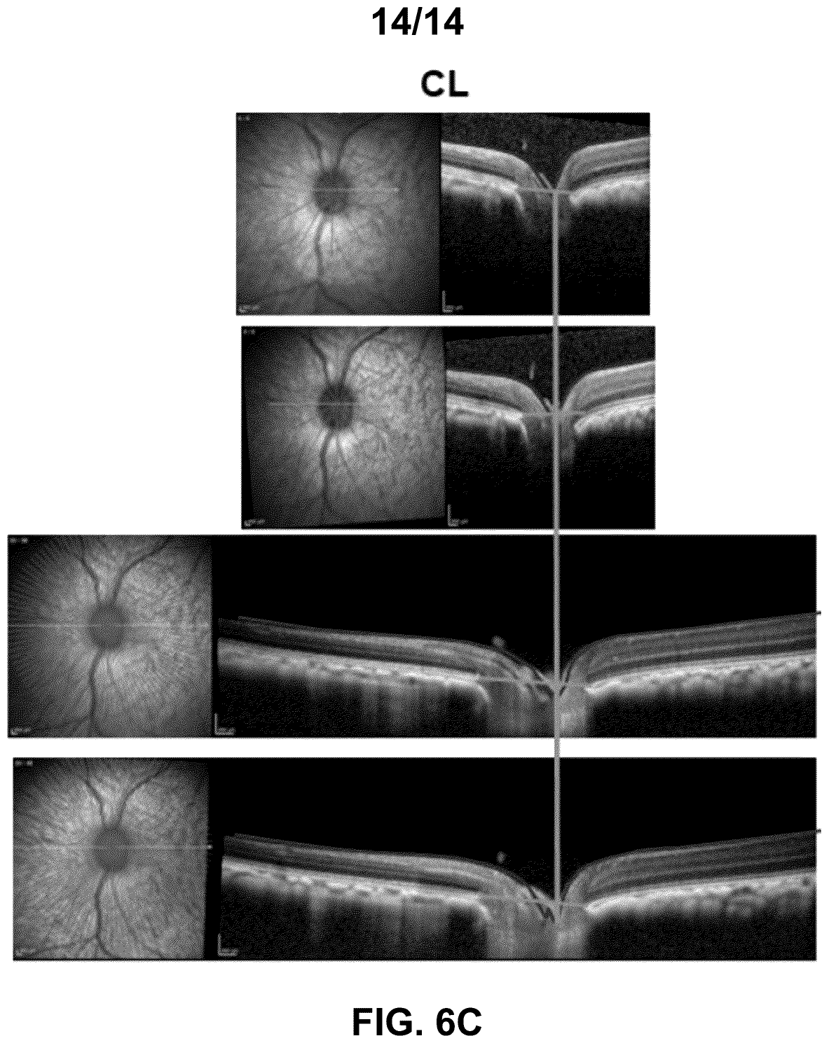

ON head “cupping” is present in the SOHU eye with persistent IOP elevation A characteristic morphological feature of human glaucoma is enlargement of the depression in the center of the ONH, called glaucomatous “cupping”. Strikingly, live fundus imaging with confocal scanning laser ophthalmoscopy (cSLO) readily detected this signature morphological change of glaucoma in the SOHU macaque eye (#38361) by at 3 and 5 mpi (FIG. 6A), corresponding to TOP elevation (FIGS. 4A-B). That ONH cupping is absent in the mouse SOHU model further confirms the similarity between macaque and human eyes. This characteristic glaucomatous optic cup enlargement was even more obvious in OCT live imaging by radial B-scan centered through the ONH (FIG. 6B). Based on previously developed measurement of the anatomic features of the macaque ONH, we applied the Visualization Toolkit (VTK) to reconstruct and delineate the OCT imaging data. We used inner limiting membrane (ILM), Bruch's membrane opening (BMO), the two discrete points at either side of the neural canal, and the BMO reference plane as references to acquire minimum rim width (MRW), rim volume (RimV), and cup volume (CupV). Obvious shortening of MRW, shrinking of RimV, and enlarging of CupV were detected in the SOHU eye compared to contralateral control eye. The H&E staining of the ONH confirmed the “cupping” phenotype and significant thinning of RNFL. The lamina cribrosa is a trabecular connective tissue to support RGC axons at the ONH. Its deformation, such as increased curve and depth, may correlate with RNFL thinning in glaucoma patients.

Interestingly, collagen staining of the ONH of the SOHU eye also showed lamina cribrosa bowing. ONH “cupping” cannot be found by fundus SLO images or OCT images in the eyes of the other macaques without persistent IOP elevation, indicating the correlation of prolonged ocular hypertension and ONH “cupping”.

Methods

Animals

The animals in this study were rhesus macaques (Macaca mulatta) born and maintained at the California National Primate Research Center (CNPRC). The CNPRC is accredited by the Association for Assessment and Accreditation of Laboratory Animal Care (AAALAC) International. Guidelines of the Association for Research in Vision and Ophthalmology Statement for the Use of Animals in Ophthalmic and Vision Research were followed. All aspects of this study were in accordance with the National Institutes of Health (NIH) Guide for the Care and Use of Laboratory Animals and all methods are reported in accordance with ARRIVE guidelines.

Phenotyping and ophthalmic examinations were performed according to an animal protocol approved by the University of California Davis Institutional Animal Care and Use Committee and Stanford University School of Medicine.

Intracameral Injection of SO

The procedure is similar to the published protocol but with modification for monkey eyes. Sedation was achieved by intramuscular injection of ketamine hydrochloride (5-30 mg/kg IM) and dexmedetomidine (0.05-0.075 mg/kg IM). The eyes were prepped in a usual sterile fashion for ophthalmic surgery including topical anesthetic 0.5% proparacaine hydrochloride (Akorn, Somerset, New Jersey) followed by 5% betadine to the ocular surface and adnexa. A disposable 15-degree blade was used to make a side-port incision at the corneal limbus to enter the anterior chamber inferiorly near the 6 o'clock position to minimize the likelihood of oil leaking out of the eye. Silicone oil (SO, 1,000 mPa·s, Silikon, Alcon Laboratories, Fort Worth, Texas) in a 3 cc syringe on a bent 25 gauge cannula was introduced into the anterior chamber. SO was injected little by little, stopping intermittently with gentle pressure applied to the posterior aspect of the limbal incision to allow for aqueous humor to exit the eye.

Oil was injected to fill the anterior chamber to a physiologic depth with roughly ˜70-80% silicone oil and to cover the entire pupil with ˜100 μl volume. After the injection, the wound was tested to insure it was self-sealing and veterinary antibiotic ointment (BNP Ophthalmic Ointment, Vetropolycin, Dechra, Overland Park, Kansas) was applied to the surface of the injected eye. The contralateral control eyes received a mock injection with no penetration of the eye. Animals were monitored by a trained technician and a veterinarian at all times.

Removing SO from the Anterior Chamber

The procedure is similar to the published protocol with modification for monkey eyes. Briefly, after the animal was anesthetized, the eye was prepped in a sterile fashion as above. A superior (12 o'clock) corneal side-port incision was made using a 15-degree blade at the corneal limbus. A 3 cc syringe filled with sterile balanced salt solution (BSS Plus, Alcon Laboratories, Ft. Worth, Texas) with a 25 gauge bent cannula was introduced into the anterior chamber and saline was gently injected little by little while periodically allowing oil to egress from the same incision by gently applying pressure to the posterior aspect of the wound. After removing all of the oil and replacing it incrementally with BSS to a physiologic depth, the cannula was removed and the wound was checked to be self-sealing, after which antibiotic ointment was applied.

Eye Examinations and Retinal Fundus Imaging

Sedated ophthalmic examination included measurement of intraocular pressure (TOP) using rebound tonometry (Icare TA0li, Finland) while the animal was held upright and with careful attention not to apply any pressure to the globe. Three TOP measurements were taken and averaged at each exam date. Examination also included pupillary light reflex testing, external and portable slit lamp examination, as well as dilated (Tropicamide 1%, Phenylephrine 2.5%, Cyclopentolate 1%) indirect ophthalmoscopy. Sedation was achieved by intramuscular injection of ketamine hydrochloride (5-30 mg/kg IM) and dexmedetomidine (0.05-0.075 mg/kg IM). Animals were monitored by a trained technician and a veterinarian at all times. Color and red-free fundus photographs were obtained with the CF-1 Retinal Camera with a 50° wide angle lens (Canon, Tokyo, Japan).

Spectral-Domain Optical Coherence Tomography (SD-OCT) Imaging

SD-OCT with confocal scanning laser ophthalmoscopy (cSLO) was also performed (Spectralis HRA+OCT, Heidelberg, Germany). High-resolution radial and circumferential scans centered on the optic nerve were obtained using a corneal curvature (K) value of 6.5 mm radius. For the high-resolution radial scans of the optic nerve head (ONH), 48 radial B-scans were acquired by 870 nm SD-OCT (Spectralis; Heidelberg Engineering, GmbH), over a 30° area, and 768 A-scans per B-scan at ART=16 repetitions. All repetitive scans were acquired using eye-tracking and averaged to reduce speckle noise. We read in all the images and measured MRW, RimV, and CupV using R program. The codes that we used to calculate MRW, RimV and CupV are at Github (HuLab-Code/ONHV). For each monkey eye, the center of the ONH was estimated and registered during the first imaging session and used to align all follow-up images. All imaging was done by the same ophthalmic imaging team. All OCT images were taken through the center of the pupil. Speculums were used and corneal hydration was maintained through application of topical lubrication (Genteal artificial tears) approximately every 1-2 minutes during imaging sessions. The en-face retinal images were captured with the Heidelberg Spectralis SLO/OCT system equipped with an 870 nm infrared wavelength light source and a 30o lens (Heidelberg Engineering). The average thickness of retinal nerve fiber layer (RNFL) around the optic nerve head was measured manually with the aid of Heidelberg software. The investigators who measured the thickness of RNFL were masked to the treatment of the samples.

Electroretinography (ERG) Recording

After dilation, a full-field ERG (ffERG) containing six different tests was performed on each eye following a 30-minute dark adaptation period. ERG-Jet electrodes (item #95-011) were coupled with the RETeval instrument (LKC Technologies, Gaithersburg, MD, United States). A standard flash electroretinogram was performed according to the approved protocol of the International Society for Clinical Electrophysiology of Vision (ISCEV). There were four dark adapted tests (0.01 cd*s/m2, 3.0 cd*s/m2, 10.0 cd*s/m2, and oscillatory potentials 3.0 cd*s/m2). After 10 minutes of light adaptation, two additional tests were performed (3.0 cd*s/m2 single flash with measurement of the photopic negative response and photopic flicker 3.0 cd*s/m2). Both time (ms) and amplitude (11V) were obtained for each test on each eye. Single flash tests measured an a-wave and b-wave.

Oscillatory potentials measured five wave points and a sum. In the photopic flicker test, the first wave point is reported. Measurements were recorded and displayed using the manufacturer's software.

Immunohistochemistry of Whole-Mount Retina and RGC Counting

The detailed procedure has been published before with modification to accommodate large monkey eyes. Briefly, after intravitreal injection with 10% formalin in PBS, the eyes and optic nerves were dissected out, post-fixed with 10% formalin for 24 hours at room temperature. Retinas were dissected out and washed extensively in PBS before blocking in staining buffer (10% normal goat serum, Sigma-Aldrich, and 2% Triton X-100 in PBS) for half an hour. RBPMS guinea pig antibody made at ProSci Inc (Poway, California) according to publications was diluted (1:4000) in the same staining buffer. Floating retinas were incubated with primary antibodies overnight at 4° C. and washed 3 times for 30 minutes each with PBS. Secondary antibodies (Cy3) were then applied (1:200; Jackson ImmunoResearch, West Grove, Pennsylvania) and incubated for 1 hour at room temperature. Retinas were again washed 3 times for 30 minutes each with PBS before a cover slip was attached with Fluoromount-G (SouthernBiotech, Birmingham, Alabama). For RGC counting, whole-mount retinas were immunostained with the RBPMS antibody, 6 fields sampled from each region (periphery, mid-periphery, and center retinas) using a 20× lens with Keyence epifluorescence microscope, and RBPMS⁺ RGCs of each image (540 μm×720 μm) were counted manually with Fiji/ImageJ. The investigators who counted the cells were masked to the treatment of the samples.

ON Semi-Thin Sections and Quantification of Surviving Axons

The ON was exposed by removing the brain and post-fixed in situ using 2% glutaraldehyde/2% PFA in 0.1M PB for 4 hours on ice. Samples were then washed with 0.1M PB 3 times, 10 minutes each wash. The ONs were then carefully dissected out and rinsed with 0.1M PB 3 times, 10 minutes each wash. They were then incubated in 1% osmium tetroxide in 0.1M PB for 1 hour at room temperature followed by washing with 0.1M PB for 10 minutes and water for 5 minutes. ONs were next dehydrated through graded ethanol, infiltrated in propylene oxide and epoxy, and embedded in epoxy at 60° C. for 24 hours. Semi-thin sections (1 μm) were cut on an ultramicrotome (EM UC7, Leica) and collected 2 mm distal to the eye. The semi-thin sections were attached to glass slides and stained with 1% para-phenylenediamine (PPD) in methanol: isopropanol (1:1) for 35 minutes. After rinsing 3 times with methanol: isopropanol (1:1), coverslips were applied with Permount Mounting Medium (Electron Microscopy Sciences, Hatfield, Pennsylvania). PPD stains all myelin sheaths, but darkly stains the axoplasm only of degenerating axons, which allows us to differentiate surviving axons from degenerating axons. The whole ON were imaged with a 100× lens of a Keyence fluorescence microscopy to cover the entire area of the ON without overlap. Four areas of 108 μm×144 μm were cropped, and the surviving axons within the designated areas counted manually with Fiji/ImageJ. After counting all the images taken from a single nerve, the mean of the surviving axon number was calculated for each ON. The investigators who counted the axons were masked to the treatment of the samples.

Anterior Segments and Retina Cross Sections and H&E and Trichrome Staining

Monkey eyes were enucleated and immediately fixed in 10% formalin for 36 hours at room temperature. They were processed through graded alcohol and xylene, then infiltrated and embedded in paraffin. Six-micron sections were taken and stained with Hematoxylin & Eosin (H&E) to look at the cell nuclei, extracellular matrix, and cytoplasm using Nikon Eclipse (E800) microscope. Standard protocol was followed to stain these slides. The Trichrome kit was purchased from Abcam (ab 150686) to study collagenous connective tissue in sections. Slides were deparaffinized and incubated in preheated Bouin's fluid for an hour and rinsed in water. They were then incubated in Weigert's Iron Hematoxylin for 5 minutes, rinsed in water again and then incubated in Biebrich Scarlet/Acid Fuchsin solution for 15 minutes. They were rinsed in water again. Sections were then differentiated in phosphotungstic acid solution for 10-15 minutes (or until collagen is not red), incubated in Aniline Blue solution for 5-10 minutes and rinsed in water. Acetic acid solution was applied to these sections for 3-5 minutes, and slides were then dehydrated in alcohol, cleared in xylene, and mounted with CytoSeal 60 (from Electron Microscopy Sciences, 18006). This stain shows a stronger collagen stain (blue green stain) in glaucomatous eye than the control eye.

Statistical Analyses

GraphPad Prism 7 was used to generate graphs and for statistical analyses. Data are presented as means±s.e.m. Student's t-test was used for two groups comparison and One-way ANOVA with post hoc test was used for multiple comparisons.

### Considerations

The present invention establishes a straightforward and minimally invasive procedure, a single intracameral injection of SO, to induce reproducible glaucomatous RGC and ON degeneration within 3-6 months in rhesus macaque monkeys. The model mimics acute secondary glaucoma caused by pupillary blocking and can be used to study the pathogenesis of neurodegeneration and to select urgently needed neuroprotectants and regeneration therapies that are unrelated to TOP management. Within 3-6 months of a simple SO intracameral injection, the SOHU eyes of all monkeys studied showed a highly consistent array of findings: significant thinning of RNFL, decreased visual function (PhNR), and loss of RGC somata and axons. The reversible intracameral SO injection does not cause overt anterior ocular structural damage other than the ciliary body while simulating acute glaucomatous RGC and ON changes. Therefore, this inducible, reproducible, and clinically relevant NHP neurodegeneration model can be used to decipher the molecular mechanisms of transient ocular hypertension-induced glaucomatous degeneration in primate, and to preclinically assess the efficacy and safety of experimental strategies for neuroprotection and regeneration.

A unique feature of this NHP model is the transient TOP elevation-induced ciliary body “shock”. Unlike mouse, but as can happen in humans, the NHP ciliary body seems very vulnerable to acutely elevated TOP, which first caused it to stop generating aqueous humor and ocular hypotension, and ultimately leads to atrophy. All six monkeys that we tested consistently developed persistent intraocular hypotension and histological evidence of ciliary body atrophy, although we captured the initial transient TOP elevation before ocular hypotension in only two animals. Unfortunately, most animals (five out of six monkeys) studied did not fully recover normal ciliary body function. Ciliary body function appeared to recover in part, however, since they became able to maintain low or normal TOP in the presence of SO-induced pupillary blocking within the time period of the experiment (3-6 months). Despite the absence of long-lasting chronic ocular hypertension, all five animals showed similar RGC and ON degeneration as the one animal with persistent ocular hypertension. This suggests that transient acute TOP elevation causes the neurodegeneration. From our mouse study, we learned that although SO removal allows TOP to return quickly to normal, it does not stop the progression of glaucomatous neurodegeneration in the SOHU model. This result is also consistent with the clinical observation that visual field loss can progress aggressively in some glaucoma patients whose TOP is maintained at a relatively low level. Thus, this NHP SOHU model can be used to determine the efficacy of experimental neuroprotection treatment when TOP is low after an initial period of pathogenic ocular hypertension, simulating clinical TOP treatment. Advanced retinal imaging and visual function assays that are available for humans can be applied to this primate glaucoma model. These assays will identify morphological and functional changes in RGCs and ON that can serve as potential biomarkers in glaucoma and other optic neuropathies. Since optic neuropathy can also be associated with other central nervous system (CNS) neurodegenerative diseases, including multiple sclerosis, Alzheimer's disease, and amyotrophic lateral sclerosis, this model may be broadly applicable to diverse CNS degenerative diseases.

One animal (#38361) was able to recover rather quickly from ciliary body shock and resume adequate aqueous humor production, which increased TOP due to pupillary blocking. It is notable that the characteristic glaucomatous ONH “cupping” was associated with persistent ocular hypertension in this animal but absent from the other animals without persistent TOP elevation.

Ocular vascular dysfunction has long been known to be correlated with the incidence of glaucoma and acutely elevated TOP in patients with angle-closure glaucoma, and secondary glaucoma can induce central retinal artery occlusion with ischemic damage of the inner retina. We have detected ocular ischemia with inner retina damage and outer retina sparing in the severe variant of the SOHU mouse model, consistent with findings in rats with acutely elevated TOP.

Interestingly, we also detected branch retinal artery occlusion in the animal (#38361) with elevated TOP, demonstrating another similarity between the ocular hypertension in NHP and the human acute glaucoma-related syndrome. We do not know what causes the variable ciliary body responses of different animals. Age may play a role since #38361 was much older (13 yrs) than the other five animals (6-8 yrs); the middle-aged ciliary body in this animal may be more resilient than younger ciliary bodies. Further systematic studies with additional senior, middle-aged, and young NHP animals are needed to clarify the reasons and to further optimize this model. For example, a modified SOHU model like the one that we developed in mouse that induces and maintains a moderate elevation of TOP through frequent pupil dilation 16 may prevent the acute severe TOP elevation causing ciliary body shock.

What is claimed is:

1. A silicone oil-induced ocular hypertension glaucoma model in a living non-human primate, comprising: an experimental eye with an iris, a pupil and an anterior chamber having in the anterior chamber a silicone oil droplet covers 70-80% iris surface, wherein the silicone oil droplet is equivalent to about 75-150 microliters and closes the anterior chamber angle, wherein the silicone oil contacts the surface of the iris such that pupillary block occurs.

2. The silicone oil-induced ocular hypertension glaucoma model as set forth in claim 1, further comprising a contralateral eye with an anterior chamber having in the anterior chamber a volume of saline which is used as a control eye relative to the experimental eye.

3. The silicone oil-induced ocular hypertension glaucoma model as set forth in claim 2, wherein the volume of saline is equivalent to about 75-150 microliters.
